(19) United States
(12) Patent Application Publication
(10) Pub. No.: US 2004/0137571 A1
(43) Pub. Date: Jul. 15, 2004

(54) Method for purifying a fermentation-derived product

(76) Inventors: Jan Markussen, Herlev (DK); Ivan Diers, Vaerlose (DK)

Correspondence Address: NOVO NORDISK PHARMACEUTICALS, INC, 100 COLLEGE ROAD WEST, PRINCETON, NY, 08540, US

(21) Appl. No.: 10/719,601

(22) Filed: Nov. 21, 2003

Related U.S. Application Data
(60) Provisional application No. 60/430,748, filed on Dec. 4, 2002.

(30) Foreign Application Priority Data
Nov. 26, 2002 (DK) PA 2002 01821

(51) Int. Cl. C07K 1/14

(52) U.S. Cl. 435/69.1; 530/412

(57) ABSTRACT

Process for purifying a fermentation-derived product.

### CROSS-REFERENCE TO RELATED APPLICATIONS

This application claims priority under 35 U.S.C. 119 of Danish application no. PA 2002 01821 filed Nov. 26, 2002 and U.S. application No. 60/430,748 filed Dec. 4, 2002, the contents of which are fully incorporated herein by reference.

### FIELD OF THE INVENTION

The present invention relates to a simple process for purification of fermentation-derived products. More specifically the processes of the invention pertain to heat treatment of culture broth for precipitation and removal of impurities.

### BACKGROUND OF THE INVENTION

The conventional method for recovering fermentation-derived products, such as proteins and antibiotics, from the complex culture broth matrix is commonly liquid chromatography. This process comprises the application of the product holding fluid onto a solid chromatographic matrix under conditions where the fermentation-derived product binds to the chromatographic matrix while the bulk of impurities pass through the chromatographic column. After a washing phase the bound product is eluted from the column. The method eliminates the major part of host cell impurities from the product.

This method also has several drawbacks. First, chromatography is an expensive method for recovery of fermentation derived products. Second, chromatography is not well suited for continuous processes which are often used in the industrial manufacture of fermentation-derived products. Third, chromatographic column operation is not robust towards normal fermentation-derived impurities such as remnant cells and cellular debris, antifoam, host cells proteins and proteases. Often many sequential steps are needed for a chromatographic recovery, including upstream centrifugation and filtration steps and several chromatographic steps each targetting a certain group of impurities.

Membrane filtration such as microfiltration and ultrafiltration has also been used for the purification steps following fermentation with some success. However, membrane filtration processes are often quite slow and relatively expensive processes.

Addition of flocculation agents has also been applied as the initial purification step for proteins (WO 96/38469 and Biotechnol. Prog. 16, 2000, 661-667), but it is expensive and gives rise to waste disposal problems.

It is a general teaching within the field of biotechnology that fermentation-derived products such as protein and antibiotics should be kept in solution at as low temperatures as possible in order to prevent microbial, enzymatic or chemical degradation of the product (Biochemical engineering fundamentals, J. E. Bailey, D. F. Ollis, McGraw-Hill Inc., 1986).

It has surprisingly been found that heat treatment of culture broth may precipitate a range of impurities without concomitantly precipitating or co-precipitating the desired product. Thus, this very simple purification method is particularly well suited for the first purification step upstream of chromatographic columns.

The present invention provides a method for the industrial manufacture of fermentation-derived products, which enables continuous manufacturing and better separation of product and impurities while reducing manufacturing costs and reducing down-time of chromatographic columns.

### SUMMARY OF THE INVENTION

The present invention provides a process for purifying a cell-derived product from a cell culture, which is carried out by an extract of the cell culture or a growth medium of the culture at a temperature between about 60° C. to 90° C. and cooling the treated extract or growth medium to a temperature below about 60° C. The high-temperature treatment may be for a period between about 1 min and about 60 min. In some embodiments, the heat treatment is for a period not more than about 30 min, 20 min, or 10 min. The cooling step may comprise cooling to a temperature below about 35° C.

In practicing the present invention, any cells may be used. In some embodiments, the cell culture comprises bacterial or yeast cells, including, without limitation, Escherichia coli, Saccharomyces cerevisiae, Pichia pastoris, Pichia methanolica, Candida utilis and Kluyveromyces lactis.

In some embodiments, the cell culture comprises recombinant cells, including, without limitation, those that have been programmed to produce a recombinant protein. Non-limiting examples of recombinant proteins include GLP-1, exendin-4, exendin-3, GLP-2, glucagon, TFF peptides, interleukins, insulin, albumin, precursors of any of the foregoing, and analogs of any of the foregoing.

### DESCRIPTION OF THE INVENTION

Fermentation derived products or precursors thereof are commonly produced by cultivation of recombinant host cells, e.g. bacteria, fungi and mammalian cells, in an appropriate fermentation medium. The fermentation medium may be chemically defined or it may be a complex medium containing the necessary nutrients for growth and product formation of the host cells, e.g. sugar, nitrogen source, salts, vitamins etc. Once the microorganism has been cultivated in the medium and the cells have optionally been disrupted, the fermentation broth contains the desired product in a mixture with remnant medium components and host cell derived impurities. Host cell derived impurities are mainly proteins, nucleic acids and, in particular where an intracellular product is released by disrupting the cells, cellular debris. The first step in the recovery or purification of the fermentation derived product is to separate the major part of the host cell derived impurities from the product and to concentrate the product.

In one aspect the present invention relates to a process for purifying a fermentation-derived product, said process comprising the steps of:

a) heating the fermentation broth containing said fermentation-derived product or a precursor thereof to a temperature in the range from 60° C. to 90° C.,

b) cooling the fermentation broth to a temperature below 60° C.;

c) separating the precipitate from the soluble portion of the fermentation broth at a temperature less than 60° C.;

d) isolating said fermentation-derived product.

The term “purifying a fermentation-derived product” as used herein means the separation of the fermentation-derived product from impurities present in the starting material. Thus, the separation results in the fermentation-derived product being of higher purity than that in the starting material.

The term “fermentation-derived product” as used herein means the product compound being produced by the overall manufacturing process. Thus, the fermentation-derived product may be a compound which is directly synthesised by the host cells, or it may be a chemical derivative or fragment of a precursor produced by the host cells. Chemical derivatives can be esters, acylated forms and PEGylated molecules.

The term “precursor” as used herein means a covalently modified form which can be converted into the desired form. If the product being produced is, for instance, a protein, then the fermentation-derived product may either be the protein itself or more often a precursor thereof. The precursor typically is the product protein with an amino acid extension which increases the yield in the fermentation process or which facilitates purification steps such as affinity chromatography, e.g. IMAC purification of his-tagged proteins. The precursor can also be the parent protein when the fermentation-derived product is a chemically modified form of the protein.

The term “fermentation broth” as used herein means the product-containing fluid which results from the fermentation process. The term “fermentation broth” encompasses solutions and suspensions, i.e. the cell free supernatant, the broth with whole cells and the broth with or without cellular debris following cell disruption as well as broth resulting from any solubilisation steps or protein refolding steps.

In a second aspect the present invention relates to a process for purifying a fermentation-derived product, said process comprising the steps of:

a) heating the fermentation broth containing said fermentation-derived product or a precursor thereof to a temperature in the range from 60° C. to 90° C.,

b) cooling the fermentation broth to a temperature below 60° C.;

c) separating the precipitate from the soluble portion of the fermentation broth at a temperature less than 60° C.;

d) isolating said fermentation-derived product;

wherein no flocculation agent is added to said fermentation broth.

The term “flocculation agent” as used herein means chemicals which are added to the fermentation broth after the fermentation has stopped in order to bind impurities forming insoluble complexes which subsequently precipitates. Examples of flocculation agents are Fe²⁺, Al³⁺ and a range of charged polymers.

In one embodiment of the process for purifying a fermentation-derived product, the soluble portion of the fermentation broth in step c) contains at least 60% of the product which results in the fermentation derived product.

In another embodiment of the process for purifying a fermentation-derived product, the pH of the fermentation broth which is heated in step a) is at least 1 pH unit, preferable at least 2 pH units from the isoelectric point of said fermentation-derived product.

In another embodiment of the process for purifying a fermentation-derived product, the mean residence time of the fermentation broth at temperatures in the range from 60° C. to 90° C. in step a) is less than 60 minutes, less than 30 minutes, less than 15 minutes, most preferable less than 10 minutes.

In a further embodiment of the process for purifying a fermentation-derived product, the fermentation broth is cooled to temperatures below 35° C. in step b).

In a further embodiment of the process for purifying a fermentation-derived product, the temperature of the fermentation broth during the separation step c) is less than 40° C., less than 35° C., less than 25° C. or less than 10° C.

In a further embodiment of the process for purifying a fermentation-derived product, the separation in step c) is performed by centrifugation. Large scale centrifuges for industrial applications are commercially available. Preferred centrifuges are for continuous operation, e.g. solids ejecting centrifuges and decanter centrifuges.

In a further embodiment of the process for purifying a fermentation-derived product, the separation in step c) is performed by microfiltration. A number of industrial scale microfiltration units are available for cross-flow microfiltration or vibrating microfiltration. Microfiltration membranes may be formed from a variety of materials such as natural polymers, synthetic polymers, ceramics and metals. Preferred microfiltration membranes are ceramic membranes which may be formed by fibres of silicon carbide, silicon nitride, aluminosilicate, mixtures thereof and which may optionally be carbon-coated (see e.g. WO 00/45938). Preferred metal microfiltration membranes are zirconium membranes.

The nominal pore size of MF membranes are typically in the range from 0.01 μm to 100 μm, preferably from 0.05 μm to 75 μm and more preferable from 0.1 μm to 50 μm. In order to prevent polarization of the membrane, the MF process is typically carried out using cross flow filtration where the broth also flows along the membrane surface.

In a further embodiment of the process for purifying a fermentation-derived product, the process steps a), b) and c) are run in continuous mode.

In a further aspect the present invention relates to a process for purifying a fermentation-derived product, said process comprising the steps of:

a) heating the fermentation broth containing said fermentation-derived product or a precursor thereof to a temperature in the range from 60° C. to 90° C.,

b) cooling the fermentation broth to a temperature below 60° C.;

c) separating of the precipitate from the soluble portion of the fermentation broth at a temperature less than 60° C.;

d) isolating said fermentation-derived product;

wherein said soluble portion of the fermentation broth produced in step c) is subjected to column chromatography.

In a further aspect the present invention relates to a process for purifying a fermentation-derived product, said process comprising the steps of:

a) heating the fermentation broth containing said fermentation-derived product or a precursor thereof to a temperature in the range from 60° C. to 90° C.,

b) cooling the fermentation broth to a temperature below 60° C.;

c) separating the precipitate from the soluble portion of the fermentation broth at a temperature less than 60° C.;

d) isolating said fermentation-derived product;

wherein said soluble portion of the fermentation broth produced in step c) is subjected to crystallization or precipitation.

In a further aspect the present invention relates to a process for purifying a fermentation-derived product, said process comprising the steps of:

a) heating the fermentation broth containing said fermentation-derived product or a precursor thereof to a temperature in the range from 60° C. to 90° C.,

b) cooling the fermentation broth to a temperature below 60° C.;

c) separating the precipitate from the soluble portion of the fermentation broth at a temperature less than 60° C.;

d) isolating said fermentation-derived product;

wherein said soluble portion of the fermentation broth produced in step c) is subjected to ultrafiltration.

In one embodiment of the process for purifying a fermentation-derived product, the cut-off value of the UF membrane is lower than four times the molecular weight of the fermentation-derived product, preferably lower than twice the molecular weight of the fermentation-derived product and most preferably lower than the molecular weight of the fermentation-derived product.

In a further embodiment of the process for purifying a fermentation-derived product, the product holding fluid resulting from said ultrafiltration is subjected to column chromatography.

In a further aspect the present invention relates to a process for purifying a fermentation-derived product, said process comprising the steps of:

a) heating the fermentation broth containing said fermentation-derived product or a precursor thereof to a temperature in the range from 60° C. to 90° C.,

b) cooling the fermentation broth to a temperature below 60° C.;

c) separating the precipitate from the soluble portion of the fermentation broth at a temperature less than 60° C.;

d) isolating said fermentation-derived product;

wherein said fermentation-derived product is a protein.

In one embodiment of the process for purifying a fermentation-derived product, said fermentation-derived product is a pharmaceutical protein or a precursor thereof.

The term “pharmaceutical protein” as used herein means a protein which has a known pharmaceutical activity.

In another embodiment of the process for purifying a fermentation-derived product, said fermentation-derived product is a commercialised pharmaceutical protein.

The term “commercialised pharmaceutical protein” as used herein means a pharmaceutical protein which has been approved by a regulatory agency in at least one country selected from US and EU countries.

In a further embodiment of the process for purifying a fermentation-derived product, said fermentation-derived product is produced by a recombinant host cell.

In a further embodiment of the process for purifying a fermentation-derived product, said host cells are selected from the group consisting of Escherichia coli, Saccharomyces cerevisiae, Pichia pastoris, Pichia methanolica, Candida utilis and Kluyveromyces lactis.

In a further embodiment of the process for purifying a fermentation-derived product, said fermentation-derived product or a precursor thereof has a molar weight of less than 25000 Dalton, less than 10000 Dalton, less than 7000 Dalton, or less than 4000 Dalton. In a further embodiment of the process for purifying a fermentation-derived product, said protein is selected from the group consisting of GLP-1, exendin-4, exendin-3, GLP-2, glucagon, TFF peptides, interleukins, insulin, albumin, precursors thereof and analogs of any of the foregoing.

In a further embodiment of the process for purifying a fermentation-derived product, said protein is Ser³⁸, Lys^{39, 40, 41, 42, 43, 44}-Exendin-4(1-39)-amide (ZP-10).

The term “analog” as used herein means a variant of a protein wherein one or more amino acid residues of the parent protein has been substituted by other amino acid residue(s) and/or wherein one or more amino acid residues have been inserted into the parent protein and/or wherein one or more amino acid residues have been deleted from the parent protein.

In one embodiment an analog differs from the parent protein in no more than five amino acid residues. In another embodiment an analog differs from the parent peptide in no more than three amino acid residues. In another embodiment an analog differs from the parent peptide in only one amino acid residue.

In a further embodiment of the process for purifying a fermentation-derived product, said protein is selected from the group consisting of human insulin, a human insulin precursor, a human insulin analog, a human insulin analog precursor, Arg³⁴-GLP-1(7-37) and GluGluAlaGluLys-Arg³⁴-GLP-1 (7-37).

### EXAMPLES

### Example 1

Heat Treatment of Fermentation Broth of Single Chain Insulin (yMaUJ95,SCI-13)

The peptide SCI-13 has the sequence: (B-chain)-Gly-Tyr-Gly-Asn-His-Asp-Leu-Asn-Phe-Pro-Gln-Thr-(A-chain), wherein (B-chain) is the 30 amino acid B-chain of human insulin, and (A-chain) is the 21 amino acid A-chain of human insulin. SCI-13 thus has a 12 amino acid peptide connecting the C-terminus of the B-chain to the N-terminus of the A-chain.

Yeast cells transformed with plasmid pMaUJ360 coding for the single chain insulin, SCI-13, were grown in a 10 L fermenter on YPD-medium with glucose added separately by a linear gradient. After 2 days fermentation 9.35 litre of broth were harvested and centrifuged to yield 7.5 litre of supernatant.

To 2 L of supernatant was added 3 L of ethanol and the pH was adjusted to 3.0 with dilute hydrochloric acid. The precipitate formed was removed by centrifugation, and 5 ml portions of the clear supernatant were subjected to treatment for 5 minutes at 60, 80 and 93° C., respectively. The amount of free SCI-13 in the samples was estimated by the following HPLC analysis:

A 4×150 mm column of C-18 5μ Licrosorb was used and the effluent analysed by UV-detection at 214 nm. A linear gradient from 90% buffer A (0.018 M (NH)₄SO₄, 0.0125 M Tris, 20% CH₃CN, pH 7.0) and 10% B (50% CH₃CN) to 20% buffer A and 80% B was applied during 20 minutes using a pumping rate of 1.5 ml/min. A standard of human insulin emerges in this system at 12.8 min and the SCI-13 compound emerges at 12.1 min.

The results of the experiment show that impurities are precipitated and that the SCI-13 compound is rendered fully soluble by the heat treatment of the broth. Thus, the solution is conditioned for further purification steps by column chromatography or other processes where it is desirable that the product is in freely soluble form.

| Temperature of treatment, ° C. for 5 min | Concentration, mg/L |
|---|---|
| None; room temperature | 0 |
| 60° C. | 3.1 |
| 80° C. | 2.3 |
| 95° C. | 2.3 |

### EXAMPLE 2

Clarification of Supernatant by Heat Treatment Before Preparative Chromatography.

Fermentation broth from yeast strain YES2507 expressing Arg³⁴-GLP-1 (7-37) with the N-terminal extension GluGluAlaGluLys (EEAEK) was prepared by fermentation as described in Example 1. The GLP-1 analog was solubilised and cells were removed by centrifugation after adjustment of the 4.2 litres of broth to pH 9.7 by adding NaOH, and pH was then quickly adjusted to 3.0 in the supernatant (3.5 litres) by addition of hydrochloric acid. The unclear and brown coloured liquid was subjected to heat treatment in a 10 litre fermentor equipped with a heating/cooling jacket. Temperature was raised from ambient to 80° C. in 3-4 minutes by injection of steam into the jacket and slow stirring of the liquid for heat transfer. The temperature was kept constant at 80° C. for 10 minutes and subsequently cooled quickly to ambient temperature by circulation of 5° C. cooling water in the jacket. The dark coloured precipitate was removed by centrifugation to give a final clear, light brown solution of 3.25 litres. This clear solution was then directly applied to a chromatography column with no further treatment. The concentration of Arg³⁴-GLP-1(7-37) in the clear solution was determined by HPLC as described in Example 1.

| | Volume | HPLC | Yield |
|---|---|---|---|
| Sample type | (L) | (mg/L/mg/L_{before heat treatment}) | (%) |
| Before heat treatment | 3.5 | 100% | 100 |
| After heat treatment | 3.25 | 82% | 76 |

### Example 3

Broth from a yeast fermentation producing GluGluAlaGluLys-Arg³⁴-GLP-1 (7-37) is collected and stored below 10° C. prior to recovery. The fermentation broth was then clarified for yeast cells by means of centrifugation. The resulting supernatant has a pH of 5.8 and a turbidity of 35 NTU units (Nephelometric Turbidity Unit). The supernatant pH is then adjusted to 3.0 by addition of sulfuric acid whereby the turbidity increases to 76 NTU. One part of the acidified supernatant is then heat treated at 80° C. for 10 minutes by passing the liquid through an heat exchanger unit using a mean residence time of 10 minutes. The heated liquid is cooled to below 10° C. once it leaves the heat exchanger. The second half of the supernatant is considered reference material and stored below 10° C.

Both the heat treated supernatant and the reference material are centrifuged and the supernatants from these centrifugations are collected. The turbidity of both ice cooled supernatants was measured to:

| | |
|---|---|
| Turbidity Heat treated supernatant: | 51 NTU |
| Turbidity reference material: | 76 NTU |

Both supernatants were stored below 10° C. for approximately 22 hours and then inspected for turbidity. The heat treated supernatant remained visually clear with NTU of 54 (ice cooled supernatant) whereas the reference material contained large fluffy, white clumps. These clumps easily disintegrated to smaller, visible particles upon shaking/stirring. The turbidity of the resulting material was measured to 72 (ice cooled material). The presence of visible particles in the reference material makes this liquid unsuited for further processing by ultrafiltration unless the particles are removed by a filtration prior to the ultrafiltration step.

1. A process for purifying a cell-derived product from a cell culture, said process comprising treating an extract of the cell culture or a growth medium of the culture at a temperature between about 60° C. to 90° C. and cooling the treated extract or growth medium to a temperature below about 60° C.

2. A process according to claim 1, further comprising subjecting the treated extract or growth medium to a separation step that separates the extract or growth medium into a precipitate and a soluble portion.

3. A process according to claim 1, wherein the treatment at a temperature between about 60° C. to 90° C. is for a period of time between about 1 min and about 60 min.

4. A process according to claim 3, wherein said time period of between about 1 min and about 30 min.

5. A process according to claim 4, wherein said time period is not more than about 10 min.

6. A process according to claim 1, wherein the extract or growth medium is cooled to a temperature below about 35° C.

7. A process according to claim 1, wherein the cell culture is selected from the group consisting of bacterial and yeast cells.

8. A process according to claim 8, wherein the cell culture is selected from the group consisting of Escherichia coli, Saccharomyces cerevisiae, Pichia pastoris, Pichia methanolica, Candida utilis and Kluyveromyces lactis.

9. A process according to claim 1, wherein the cell culture comprises recombinant cells.

10. A process according to claim 1, wherein the cell-derived product is a protein.

11. A process according to claim 10, wherein the protein has a molecular weight below about 25,000 daltons.

12. A process for purifying a fermentation-derived product, said process comprising the steps of:
a) heating the fermentation broth containing said fermentation-derived product or a precursor thereof to a temperature in the range from 60° C. to 90° C.,
b) cooling the fermentation broth to a temperature below 60° C.;
c) separating the precipitate from the soluble portion of the fermentation broth at a temperature less than 60° C.; and
d) isolating said fermentation-derived product.

13. The process according to claim 12, wherein no flocculation agent is added to said fermentation broth.

14. The process according to claim 12, wherein the temperature of the fermentation broth during the separation step c) is less than about 40° C.

15. The process according to claim 12, wherein separation in step c) is performed by a method selected from the group consisting of centrifugation, microfiltration, and combinations of any of the foregoing.

16. The process according to claim 12, wherein the process steps a), b) and c) are run in continuous mode.

17. The process according to claim 12, wherein said soluble portion of the fermentation broth produced in step c) is subjected to a method selected from the group consisting of column chromatography; crystallization; precipitation; ultrafiltration; or combinations of any of the foregoing.

18. The process according to claim 12, wherein the cut-off value of the UF membrane is lower than about four times the molecular weight of the fermentation-derived product.

19. The process according to claim 12, wherein said fermentation-derived product or a precursor thereof is a protein.

20. The process according to claim 19, wherein said protein is selected from the group consisting of GLP-1, exendin-4, exendin-3, GLP-2, glucagon, TFF peptides, interleukins, insulin, albumin, precursors of any of the foregoing, and analogs of any of the foregoing.

21. The process according to claim 20, wherein said protein is selected from the group consisting of human insulin, a human insulin precursor, a human insulin analog, a human insulin analog precursor, Arg³⁴-GLP-1 (7-37), and GluGluAlaGluLys-Arg³⁴-GLP-1(7-37).
